(19) United States
(12) Patent Application Publication
(10) Pub. No.: US 2005/0197644 A1
(43) Pub. Date: Sep. 8, 2005

(54) Colon hydrotherapy device

(76) Inventor: Challen W. Waychoff II, Piedmont, OH (US)

Correspondence Address: CALFEE, HALTER & GRISWOLD, LLP, 1110 FIFTH THIRD CENTER, 21 EAST STATE STREET, COLUMBUS, OH, 43215-4243, US

(21) Appl. No.: 10/792,086

(22) Filed: Mar. 3, 2004

(51) Int. Cl. A61M 31/00

(52) U.S. Cl. 604/514; 604/275

(57) ABSTRACT

A colon hydrotherapy device that includes a multi-chambered housing, a specially designed nozzle for creating a water vortex within the subject's colon for removing impacted fecal matter, and an insertion rod for facilitating the insertion of the device into the subject.

### STATEMENT REGARDING FEDERALLY FUNDED RESEARCH

This invention was not made by an agency of the United States Government nor under contract with an agency of the United States Government.

### TECHNICAL FIELD OF THE INVENTION

This invention relates generally to devices and methods for providing and performing colon hydrotherapy, and more specifically to a multi-chambered device for providing separate passageways for fluid into and out of the human body.

### BACKGROUND OF THE INVENTION

For the sake of improving an individual's general health, the tissues of the lower intestine may be cleansed with water using a process known historically to those skilled in the art as colonic irrigation or, more recently, as colon hydrotherapy. This process provides an effective means for removing impacted fecal matter and other undesired material from the subject. In general, colon hydrotherapy is performed as a preventive measure to sustain healthy digestion, peristalsis, and bowel tissue.

Colonic irrigation has been available from health care professionals for many years, and numerous devices for use in performing this procedure are known in the art. Typically, the subject lies on his or her back or side and a tube known as a speculum is inserted into the rectum. Fresh water is directed into the subject's bowel, thereby loosening waste material that may have accumulated on the walls of the colon and allowing this loosened material to flow out of the subject's colon. With prior art devices, the fresh water usually enters the body through the same internal passageway in the speculum that is utilized to remove the waste material. This configuration is disadvantageous because it permits an undesirable mixing of the fresh water and the waste material. Thus, there is a need for a speculum that provides separate internal passages or chambers for fresh water input and waste water drainage.

The effectiveness of colonic irrigation often depends on the water pressure, volume, and continuous flow that a given prior art device or system is capable of achieving. The complete or nearly complete removal of impacted fecal material may require a water pressure, velocity, or continuous flow that most prior art devices are incapable of delivering. Thus, there is a need for a colonic irrigation device that provides a high-volume, high-pressure or high-velocity delivery of water that is capable of effectively removing impacted material and other debris from the subject's colon.

### SUMMARY OF THE INVENTION

These and other deficiencies of the prior art are overcome by the present invention, the exemplary embodiment of which provides colon hydrotherapy device that includes a multi-chambered housing, a specially designed nozzle for creating a water vortex, and a means for creating a continuous flow of water into and out of the subject of the hydrotherapy.

In the exemplary embodiment, the housing is substantially cylindrical in shape and includes an anterior portion, a tapered posterior portion connected to the anterior portion, a stem attached to the exterior of the housing, a first internal chamber extending completely through the stem and through the length of the housing, and a second internal chamber extending from the posterior portion of the housing to the anterior portion of the housing. The second chamber is separate and distinct from the first chamber.

A nozzle is attached to the housing, and further includes a primary water inlet in communication with a plurality of water outlets that create a water vortex when a pressurized stream of water is passed into and through the device. Also provided is an insertion rod for facilitating insertion of the device into the subject's colon. The insertion rod includes a rounded tip at one end, a groove in the rounded tip that corresponds to the primary water inlet in the nozzle, and a planar grasping member at the end of the insertion rod opposite the rounded tip for removing the insertion rod after the device has been inserted into the subject.

Further advantages of the present invention will become apparent to those of ordinary skill in the art upon reading and understanding the following detailed description of the preferred embodiments.

### BRIEF DESCRIPTION OF THE DRAWINGS

The accompanying drawings, which are incorporated into and form a part of the specification, schematically illustrate one or more exemplary embodiments of the invention and, together with the general description given above and detailed description of the preferred embodiments given below, serve to explain the principles of the invention.

FIG. 1a is a perspective view of the assembled colon hydrotherapy device showing the appearance of the device prior to insertion into the body.

FIG. 1b is a perspective view of the device of FIG. 1 showing the appearance of the device after insertion of the device into the body, wherein the insertion rod has been removed and the water input and drainage lines have been attached to the housing.

FIG. 2 is an exploded, perspective view of the device of FIG. 1 showing the preferred arrangement of the various components of the invention relative to one another.

FIG. 3a is side view of the device of FIG. 1 device showing the appearance of the assembled device prior to insertion into the body.

FIG. 3b is a cross-sectional side view of the device of FIG. 3a showing the separate interior chambers for water inflow and outflow.

FIG. 4a is a cross-sectional front view of the device of FIG. 1, showing the internal structure of the nozzle and the anterior portion of the assembled device.

FIG. 4b is a cross-sectional front view of the device of FIG. 1, showing the internal structure further down the anterior portion of the assembled device near the stem.

FIG. 5a is a perspective, rear-view of the nozzle portion of the colon hydrotherapy device of FIG. 1.

FIG. 5b is a perspective, front-view of the nozzle portion of the colon hydrotherapy device of FIG. 1.

FIG. 5c is a side view of the nozzle portion of the colon hydrotherapy device of FIG. 1 showing a cutaway view of one of the water jet outlets.

FIG. 5d is a rear view of the nozzle portion of the device of FIG. 1.

FIG. 5e is a front view of the nozzle portion of the device of FIG. 1.

### DETAILED DESCRIPTION OF THE INVENTION

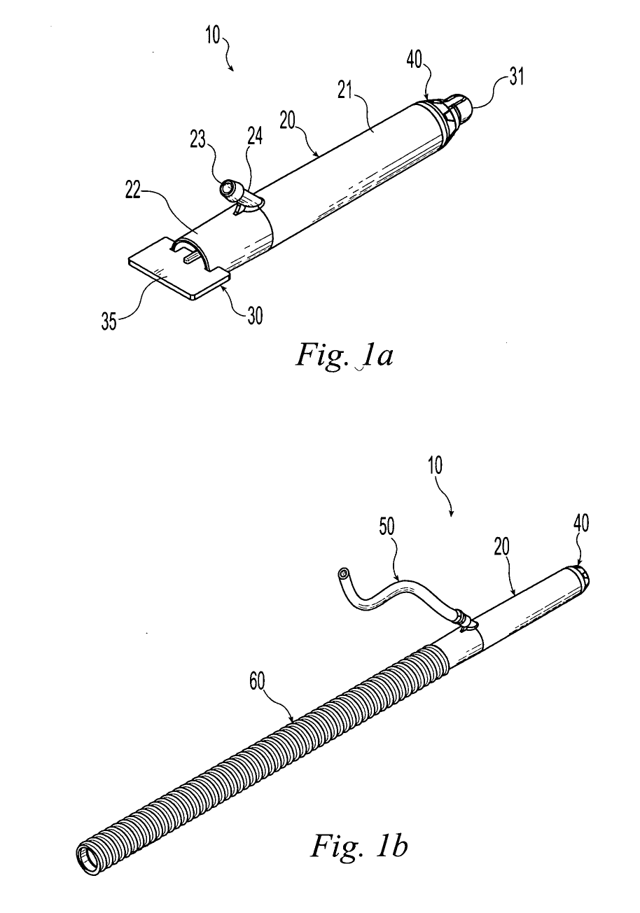

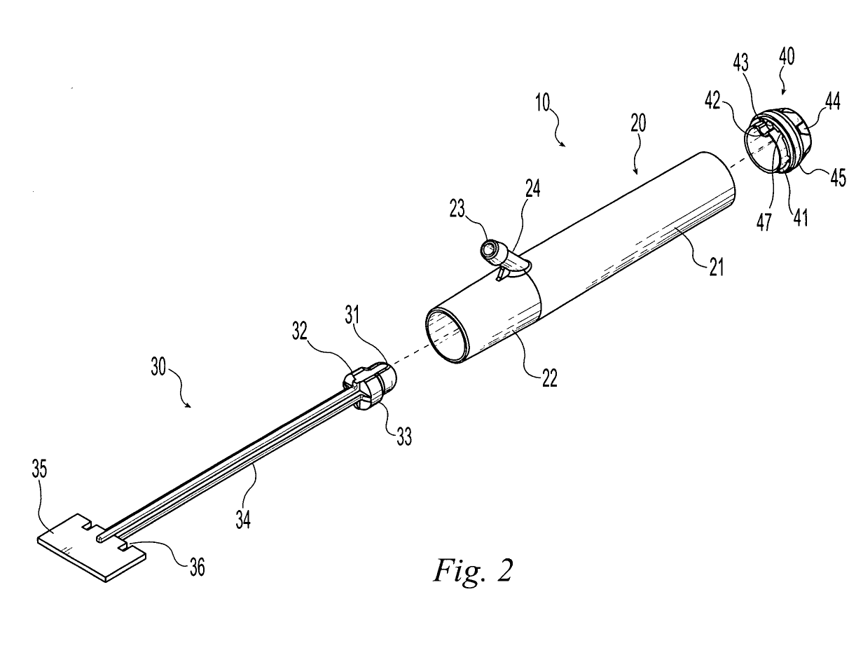

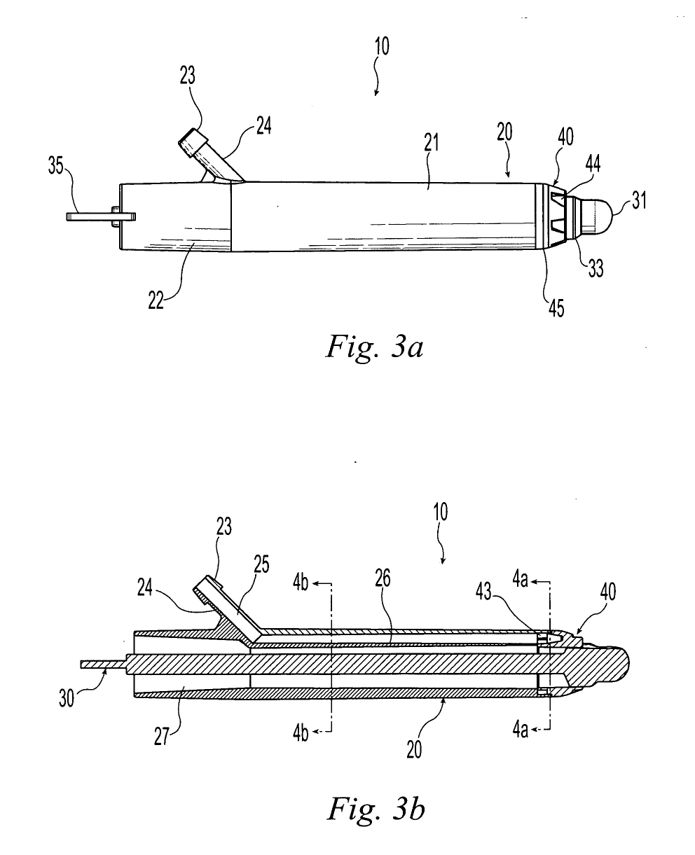

The present invention provides a highly effective device and method for performing human colon hydrotherapy. With reference to the Figures, FIG. 1a shows a perspective rear view of an exemplary embodiment of assembled colon hydrotherapy device 10 prior to insertion of the device into the body. FIG. 1b provides a perspective view of the device after insertion into the body, and FIG. 2 provides an exploded, perspective rear view of the device. FIGS. 3a and 3b provide side and cross-sectional views of the device. The device of the present invention may be fabricated using techniques known in the art, and may be manufactured from plastic, polymer, or comparable materials or combinations of such materials.

As best shown in FIG. 2, the exemplary embodiment of the device includes a speculum or housing 20 that is substantially cylindrical in shape and includes an open-ended anterior portion 21 and an open-ended posterior section 22. Posterior section 22 tapers slightly toward its open end to facilitate the attachment of drainage line 60 to the housing as shown in FIG. 2. Stem 24 is located on the upper portion of the housing and bridges both the anterior and posterior portions of the housing. Flange 23 is formed at the end of stem 24 and facilitates the attachment of water input line 50 (see FIG. 1b) to the device.

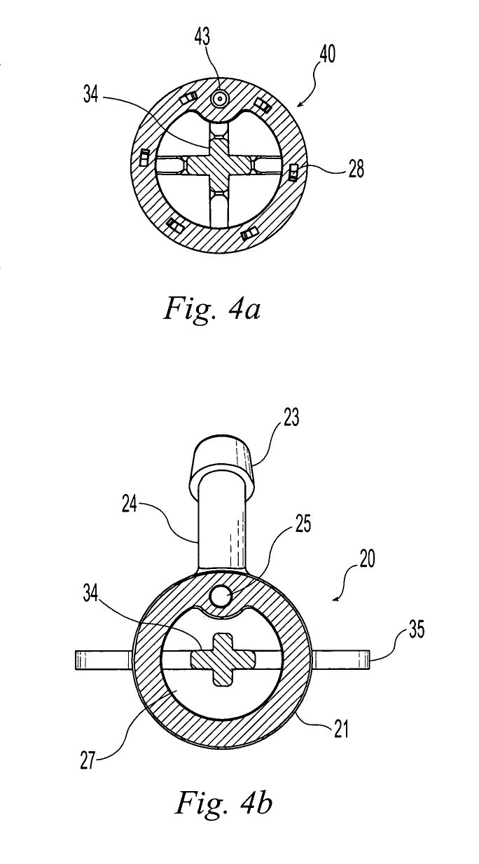

With reference to FIGS. 3b and 4b, housing 20 also includes multiple internal passages for the continuous inflow and outflow of water or other fluid while the device is in operation. First interior chamber 25 is in fluid communication with stem 24 and provides a means for water entering the device to travel through the housing, into the nozzle, and out of the device and into the body. Second internal chamber 27 runs the entire length of the housing and provides a means for wastewater and solid matter, i.e., effluent, to exit the body. Dividing wall 26 separates the two passages on the interior of housing 20. A primary advantage of this device configuration is that liquid and solid matter exiting the body during treatment does not contaminate the clean water entering the body. Furthermore, the separate chambers within the housing permit the flow of water or other fluid through the device and into the body and then out of the body through the device to be substantially continuous and uninterrupted throughout any given hydrotherapy session.

With reference to FIGS. 2 and 3a, insertion component 30 includes obturetor 31, rod 34, and grasping member 35. Obturetor 31 includes a relatively smooth, rounded tip that facilitates the insertion of colon hydrotherapy device 10 into the human body by way of the rectum. Groove 32 is a concave structure that corresponds to the convex underside of channel 42 and allows the insertion component to fit properly when nozzle 40 is attached to housing 20. Stabilization member 33 is formed behind obturetor 31 and allows the obturetor to fit securely within nozzle 40. Rod 34 extends between obturetor 31 and planar grasping member 35 and provides rigid, structural support to the entire insertion component. Grasping member 35 includes at least one notch 36 that allows gasping member 35 to fit securely against anterior portion 22, while grasping member 35 provides a means by which insertion component may be grasped and removed from housing 20 and discarded once device 10 has been inserted into the body. When the obturetor is removed, waste drainage line 60 is connected to the posterior portion of the housing and waste matter then is eliminated through the opening that contained the obturetor, through the drainage line and into the sewer.

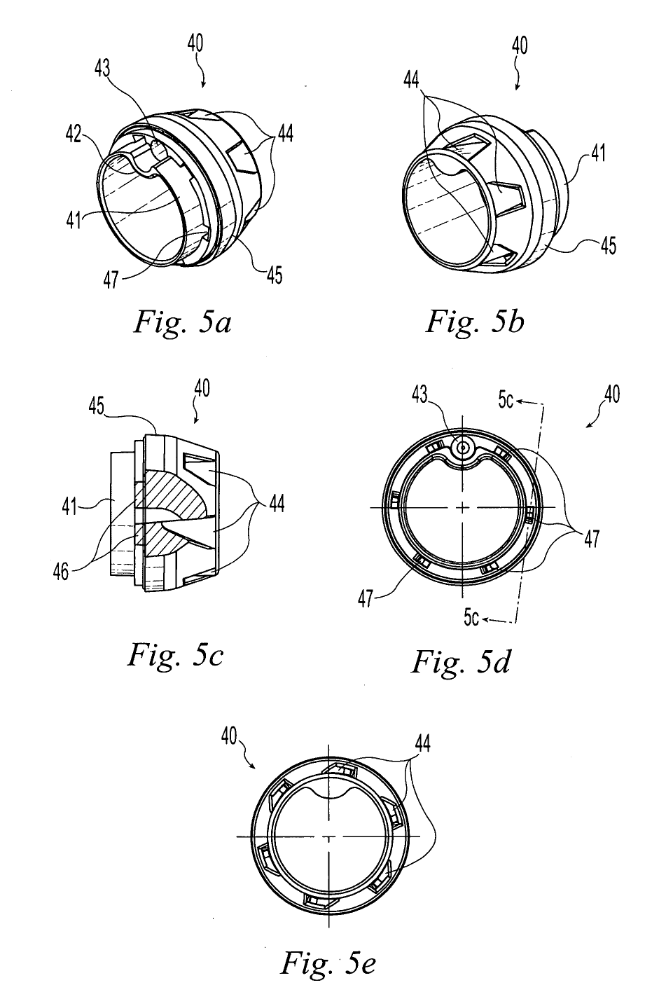

With reference to FIGS. 2, and 5a-e, nozzle 40 provides a means for directing clean treatment water into the human body after colon hydrotherapy device 10 has been inserted. In the exemplary embodiment shown in the Figures, nozzle 40 is permanently attached to the open end of anterior portion 21 of housing 20. Posterior ring 41 is of a lesser diameter than the open end of anterior portion 21, thereby allowing nozzle 40 to be inserted into housing 20. Channel 42 is formed in posterior ring 41 and its geometry corresponds to that of groove 32, which is formed in obturetor 31. Aperture 43 is formed in the rear portion of anterior ring 45, and when the nozzle is properly attached to and flush with the housing, aperture 43 is in fluid communication with first interior chamber 25. Water enters nozzle 40 through aperture 43 enters a series of internal channels 46 and exits the nozzle through a plurality of outlets 44 that are formed at regular intervals in the anterior portion of the nozzle (see FIG. 5e).

Preferably, nozzle 40 is permanently attached to housing 20 using a technique known in the art as ultrasonic welding. A plurality of projections 28 formed on anterior portion 21 of the housing corresponds to a plurality of notches 47 on the rear surface of the nozzle. The projections are inserted into the notches to define the weld zone on the device. The projections are then flexed by an oscillating force at rates of about 10,000 to 70,000 times per second (kHz), causing them to melt and flow across the joint to create a weld. A metal tool that is oscillating vertically contacts the plastic components at a distance from the projections and is the means for delivering the energy to the weld zone. The projections 28 may be a thermoplastic polymer or copolymer such as polypropylene or acrylonitrile butadiene styrene (ABS).

The present invention is designed to wash the large intestine with a continual stream of water, delivering from about 15 to 100 gallons of water in a period of about 45 minutes. The volume of water delivered to the body is dependent on the pressure output of the device. To effectively dissolve or break-up solid matter found within the large intestine, nozzle 40 creates a high-pressure water vortex (i.e., spiral) as water flows into device 10, into nozzle 40, and out of the nozzle through water outlets 44. The spiral motion of the water vortex created by nozzle 40 washes the walls of colon upon entry into the body and loosens the fecal matter found therein; the central portion of the vortex then creates suction or a vacuum that draws debris out of the body, into second interior chamber 27, and out of the body through drainage line 60. The pressurized flow of water created by device 10 also permits “pounding” or “breaking” of any large material being discharged so that this material is not able to plug or stop the effluent hole in posterior portion 22 of housing 20.

While the above description contains much specificity, this should not be construed as a limitation on the scope of the invention, but rather as an exemplification of certain preferred or exemplary embodiments. Numerous other variations of the present invention are possible, and it is not intended herein to mention all of the possible equivalent forms or ramifications of this invention. Various changes may be made to the present invention without departing from the scope or spirit of the invention

1. A colon hydrotherapy device, comprising:
(a) a housing, wherein said housing is adapted to receive fluid inflow, and wherein said housing further comprises a first internal chamber extending substantially through the length of said housing for fluid inflow and a second internal chamber distinct from said first chamber for fluid outflow; and
(b) a nozzle attached to said housing, wherein said nozzle further comprises a plurality of water outlets for creating a fluid vortex when a pressurized stream of fluid is passed through said device.

2. The device of claim 1, further comprising an insertion rod residing in said housing for facilitating the insertion of said device into a subject.

3. A colon hydrotherapy device, comprising:
(a) a housing, wherein said housing is substantially cylindrical in shape and further comprises:
(i) an anterior portion;
(ii) a tapered posterior portion connected to said anterior portion;
(iii) a stem on the exterior of said housing;
(iv) a first internal chamber extending through said stem and substantially through the length of said housing; and
(v) a second internal chamber extending from said posterior portion of said housing to said anterior portion of said housing, and wherein said second chamber is separate and distinct from said first chamber; and
(b) a nozzle attached to said housing, wherein said nozzle further comprises a primary water inlet in communication with a plurality of water outlets for creating a water vortex when a pressurized stream of water is passed through said device.

4. The device of claim 3, further comprising an insertion rod for facilitating insertion of said device into said colon, said insertion rod further comprising:
(a) a rounded tip at one end of said insertion rod;
(b) a groove in said rounded tip corresponding to said primary water inlet in said nozzle; and
(c) a planar grasping member at the end of said insertion rod opposite said tip, said grasping member further comprising at least one notch for stabilizing said insertion rod within said device.

5. The system of claim 3, further comprising a source of pressurized water.

6. The system of claim 3, further comprising a water input line attached to said stem.

7. The system of claim 3, further comprising a drainage line attached to said housing.

8. A colon hydrotherapy device, comprising:
(a) a housing, wherein said housing is substantially cylindrical in shape and further comprises:
(i) an anterior portion;
(ii) a tapered posterior portion connected to said anterior portion;
(iii) a stem on the exterior of said housing;
(iv) a first internal chamber extending through said stem and substantially through the length of said housing; and
(v) a second internal chamber extending from said posterior portion of said housing to said anterior portion of said housing, and wherein said second chamber is separate and distinct from said first chamber; and
(b) a nozzle attached to said housing, wherein said nozzle further comprises a primary water inlet in communication with a plurality of water outlets for creating a water vortex when a pressurized stream of water is passed through said device; and
(c) an insertion rod for facilitating insertion of said device into said colon, said insertion rod further comprising:
(i) a rounded tip at one end of said insertion rod;
(ii) a groove in said rounded tip corresponding to said primary water inlet in said nozzle; and
(iii) a planar grasping member at the end of said insertion rod opposite said tip, said grasping member further comprising at least one notch for stabilizing said insertion rod within said device.

9. The device of claim 8, further comprising a source of pressurized water.

10. The device of claim 8, further comprising a water input line attached to said stem.

11. The device of claim 8, further comprising a drainage line attached to said tapered posterior portion of said housing.

12. A method for performing colon hydrotherapy on a subject, said method comprising the steps of:
(a) attaching a colon hydrotherapy device to a source of pressurized water, wherein said colon hydrotherapy device further comprises:
(i) a housing, wherein said housing is adapted to receive water inflow, and wherein said housing further comprises a first internal chamber extending substantially through the length of said housing for water inflow and a second internal chamber distinct from said first chamber for water outflow; and
(ii) a nozzle attached to said housing, wherein said nozzle further comprises a plurality of water outlets for creating a water vortex when said pressurized water is passed through said device; and
(iii) an insertion rod for facilitating insertion of said device into said colon; and
(b) inserting said device into the colon of said subject by way of the rectum, wherein said insertion is performed by said subject;
(c) removing said insertion rod from said device;
(d) attaching an outflow line to said housing; and
(e) running said pressurized water through said device.

13. The method of claim 12, wherein said water enters said subject at a volume of about 15 to about 100 gallons in a time period of about 45 minutes.
